(19) United States
(12) Patent Application Publication
(10) Pub. No.: US 2008/0200504 A1
(43) Pub. Date: Aug. 21, 2008

(54) Amide Alkyl Pyridiyl Quinolines as Nk3 Receptor Modulators

(75) Inventors: Jeffrey S. Albert, Wilmington, DE (US); Cristobal Alhambra, Wilmington, DE (US); James Kang, Wilmington, DE (US); Gerard M. Koether, Wilmington, DE (US); Thomas R. Simpson, Wilmington, DE (US); James Woods, Wilmington, DE (US); Yan Li, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/063,143

(22) PCT Filed: Aug. 9, 2006
(86) PCT No.: PCT/SE2006/000934

§ 371 Date: Feb. 7, 2008

Related U.S. Application Data
(60) Provisional application No. 60/707,275, filed on Aug. 11, 2005.

(51) Int. Cl. A61K 31/47; C07D 215/00; A61P 25/00; A61P 3/00; A61P 1/00; A61P 35/00

(52) U.S. Cl. 514/314; 546/169

(57) ABSTRACT

Compounds of Formula (I) wherein R¹, A, R², R³, R⁴, R⁵,n, m and q are as described in the specification, pharmaceutically-acceptable salts, methods of making, pharmaceutical compositions containing and methods for using the same.

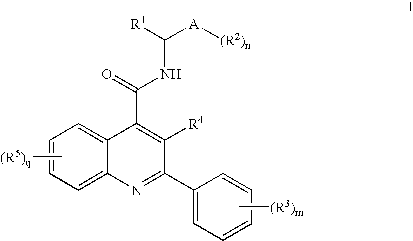

### FIELD OF THE INVENTION

This invention relates to quinoline derivatives, pharmaceutical compositions comprising them, and the use of such compounds in the treatment of central nervous system and peripheral diseases or disorders. This invention also relates to the use of such compounds in combination with one or more other CNS agents to potentiate the effects of the other CNS agents. The compounds of this invention are also useful as probes for the localization of cell surface receptors.

### BACKGROUND OF THE INVENTION

Tachykinin receptors are the targets of a family of structurally related peptides which include substance P (SP), neurokinin A. (NKA) and neurokinin B (NKB), collectively “tachykinins.” Tachykinins are synthesized in the central nervous system (CNS), and peripheral tissues, where they exert a variety of biological activities. Three tachykinin receptors are known which are named neurokinin-1 (NK-1), neurokinin-2 (NK-2) and neurokinin-3 (NK-3) receptors. NK-1 and NK-2 receptors are expressed in a wide variety of peripheral tissues and NK-1 receptors are also expressed in the CNS whereas NK-3 receptors are primarily expressed in the CNS.

The neurokinin receptors mediate a variety of tachykinin-stimulated biological effects that include: transmission of excitatory neuronal signals in the CNS and periphery (e.g. pain signals), modulation of smooth muscle contractile activity, modulation of immune and inflammatory responses, induction of hypotensive effects via dilation of the peripheral vasculature, and stimulation of endocrine and exocrine gland secretions.

In the CNS, activation of NK-3 receptors has been shown to modulate dopamine, acetylcholine and serotonin release, suggesting a therapeutic utility for NK-3 ligands for the treatment of a variety of disorders including anxiety, depression, schizophrenia and obesity. Studies in primate brain have shown the presence of NK-3 mRNA in a variety of regions relevant to these disorders. Studies in rats have shown NK-3 receptors to be located on MCH-containing neurons in the lateral hypothalamus and zona incerta, again suggesting a therapeutic utility for NK-3 ligands for obesity.

Non-peptide ligands have been developed for each of the tachykinin receptors, however known non-peptide NK-3 receptor antagonists suffer from a number of problems such as species selectivity which limits the potential to evaluate these compounds in many appropriate disease models. New non-peptide NK-3 receptor ligands are therefore desirable for use as therapeutic agents and as tools to investigate the biological consequences of NK-3 receptor modulation.

### DESCRIPTION OF THE INVENTION

Disclosed are compounds, particularly quinoline derivatives with affinity for NK-3 receptors (NK-3r). These compounds have potential for the treatment of a broad array of diseases, disorders and conditions including but not limited to depression, anxiety, schizophrenia, cognitive disorders, psychoses, obesity, inflammatory diseases including irritable bowel syndrome and inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, and testicular cancer in which modulation of the activity of NK-3 receptors is beneficial.

Ligands for NK-3 receptors disclosed and stereoisomers, enantiomers, in vivo-hydrolysable precursors and pharmaceutically-acceptable salts thereof are compounds of Formula I,

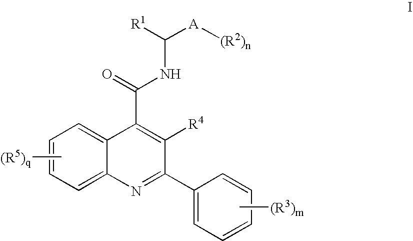

wherein:

R¹ is selected from H, C₁₋₄alkyl-, C₃₋₆cycloalkyl- and C₁₋₄alkylOC(O)-;

A is pyridyl;

R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;

n is 1, 2 or 3;

R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;

m is 1, 2 or 3;

R⁴ is selected from H, —OH, —NH₂, —OSO₂R⁶, C₁₋₄alkyl-, C₁₋₄alkoxy-, C₁₋₆alkoxyC₁₋₆alkyl-, and E-(CH₂)ₚ-, where E is selected from —NR⁶R⁷, —SOC₁₋₆alkyl, —SO₂C₁₋₆alkyl, —NR⁶SO₂R⁷, —SR⁶, N⁺(O⁻)R⁶R⁷, aryl and an N- or C-linked 5- or 6-membered aromatic or non-aromatic heterocyclic ring having 1, 2, 3 or 4 nitrogen atoms or an N-oxide thereof, and p is 0, 1, 2, 3, 4 or 5;

R⁵ at each occurrence is independently selected from H, —OH, —CN, halogen, —R⁶, —OR⁶, —NR⁶R⁷, —SR⁶, —SOR⁶ and —SO₂R⁶;

q is 1, 2 or 3;

wherein:

R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;

and,

when R⁴ is E-(CH₂)ₚ- and said E thereof is an N or C linked 5- or 6-membered aromatic or non-aromatic heterocyclic ring or an N-oxide thereof, said E is unsubstituted or has 1, 2 or 3 substituents independently selected from —OH, ═O, —NH₂, —CN, halogen, C₁₋₄alkyl-, C₁₋₄alkoxy-, C₁₋₄alkyl-CO—, —NR⁶R⁷, aryl and a 5- or 6-membered aromatic or non-aromatic heterocyclic ring having 1, 2, 3 or 4 nitrogen atoms;

and,

when R¹, R², R³ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen.

Also disclosed are pharmaceutical compositions and formulations containing the compounds, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes. In particular are disclosed compounds, compositions containing them, and methods using them for treating or preventing conditions and disorders associated with a wide range of diseases or disorders in which NK-3 receptors are considered to have a role.

### DETAILED DESCRIPTION OF THE INVENTION

Compounds of the invention are compounds of Formula I.

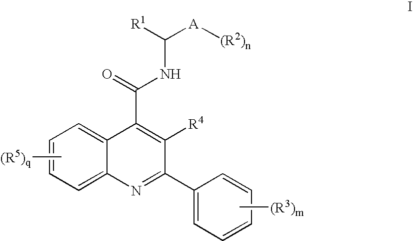

wherein:

R¹ is selected from H, C₁₋₄alkyl-, C₃₋₆cycloalkyl- and C₁₋₄alkylOC(O);

A is pyridyl; :

R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;

n is 1, 2 or 3;

R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;

m is 1, 2 or 3;

R⁴ is selected from H, —OH, —NH₂, —OSO₂R⁶, C₁₋₄alkyl-, C₁₋₄alkoxy-, C₁₋₆alkoxyC₁₋₆alkyl-, and E-(CH₂)ₚ-, where E is selected from —NR⁶R⁷, —SOC₁₋₆alkyl, —SO₂C₁₋₆alkyl, —NR⁶SO₂R⁷, —SR⁶, N⁺(O⁻)R⁶R⁷, aryl and an N- or C-linked 5- or 6-membered aromatic or non-aromatic heterocyclic ring having 1, 2, 3 or 4 nitrogen atoms or an N-oxide thereof, and p is 0, 1, 2, 3, 4 or 5;

R⁵ at each occurrence is independently selected from H, —OH, —CN, halogen, —R⁶, —OR⁶, —NR⁶R⁷, —SR⁶, —SOR⁶ and —SO₂R⁶;

q is 1, 2 or 3;

wherein:

R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═I, —NH₂, —CN, halogen, aryl and C₁₋₃alkoxy-;

and,

when R⁴ is E-(CH₂)ₚ- and said E thereof is an N or C linked 5- or 6-membered aromatic or non-aromatic heterocyclic ring or an N-oxide thereof, said E is unsubstituted or has 1, 2 or 3 substituents independently selected from —OH, ═O, —NH₂, —CN, halogen, C₁₋₄alkyl-, C₁₋₄alkoxy-, C₁₋₄alkyl-CO—, —NR⁶R⁷, aryl and a 5- or 6-membered aromatic or non-aromatic heterocyclic ring having 1, 2, 3 or 4 nitrogen atoms;

and,

when R¹, R², R³ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen;

stereoisomers, enantiomers, in vivo-hydrolysable precursors and pharmaceutically-acceptable salts thereof.

Particular compounds of the invention are those of Formula I wherein:

A is selected from pyrid-2-yl, pyrid-3-yl and pyrid-4-yl;

R¹ is selected from C₁₋₄alkyl-, C₃₋₆cycloalkyl- and C₁₋₄alkylOC(O)-;

R² is selected from H, halogen and unsubstituted C₁₋₆alkoxy-;

R³ is H or halogen;

n and m are both 1, and when R¹ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN and halogen;

stereoisomers, enantiomers, in vivo-hydrolysable precursors and pharmaceutically-acceptable salts thereof.

Other particular compounds of the invention are those of Formula I wherein:

A is selected from pyrid-2-yl, pyrid-3-yl and pyrid-4-yl;

R¹ is selected from C₁₋₄alkyl- and C₃₋₆cycloalkyl-;

R2 is selected from H, halogen and unsubstituted C₁₋₆alkoxy-;

R³ is H or halogen;

n and m are both 1;

R4 is selected from H, —OH, —NH₂, C₁₋₄alkyl-, C₁₋₄alkoxy- and E-(CH₂)ₚ-, where E is a substituted or unsubstituted N-linked 5- or 6-membered aromatic or non-aromatic heterocyclic ring having 1, 2, 3 or 4 nitrogen atoms, and R⁵ is H;

stereoisomers, enantiomers, in vivo-hydrolysable precursors and pharmaceutically-acceptable salts thereof. Still other particular compounds are those wherein:

A is selected from pyrid-2-yl, pyrid-3-yl and pyrid-4-yl;

R¹ is ethyl or cyclopropyl;

R² is selected from H, F and —OCH₃;

R³ is H or F;

n, m and q are each 1;

R⁴ is selected from H, —OH, —CH₃, —OCH₃, and NH₂, and

R⁵ at each occurrence is independently selected from H, —OH and halogen;

stereoisomers, enantiomers, in vivo-hydrolysable precursors and pharmaceutically-acceptable salts thereof.

Still other particular compounds are enantiomers in accord with Formula II

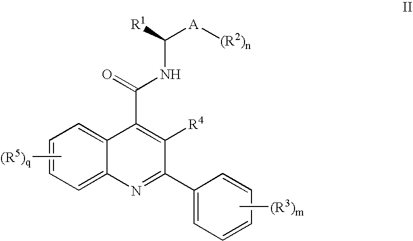

wherein R¹, A, R², n, R³, m, R⁴, R⁵ and q are as defined for Formula I;

stereoisomers, enantiomers, in vivo-hydrolysable precursors and pharmaceutically-acceptable salts thereof.

Compounds of the present invention have the advantage that they may be more soluble, be more easily absorbed and more efficacious in vivo, produce fewer side effects, be less toxic, be more potent, more selective, be longer acting, be less metabolized and/or have a better pharmacokinetic profile than, or have other useful pharmacological or physicochemical properties over known compounds. Using assays for functional activity described herein, compounds of the invention will be found to have IC50's of less than about 1 μM for NK-3 receptors and many compounds will be found to have IC50's of less than about 100 nM for NK-3 receptors.

### ABBREVIATIONS AND DEFINITIONS

As used herein, unless otherwise indicated, C₁₋₆alkyl includes but is not limited to methyl, ethyl, n-propyl, n-butyl, i-propyl, i-butyl, t-butyl, s-butyl moieties, whether alone or part of another group and alkyl groups may be straight-chained or branched.

As used herein, unless otherwise indicated, C₁₋₆alkoxy includes but is not limited to —O-methyl, —O-ethyl, —O-n-propyl, —O-n-butyl, —O-i-propyl, —O-i-butyl, —O-t-butyl, —O-s-butyl moieties, whether alone or part of another group and alkoxy groups may be straight-chained or branched.

As used herein C₃₋₆cycloalkyl groups include but are not limited to the cyclic alkyl moieties cyclopropyl, cyclobutyl, cyclopentyl and cyclohexyl.

As used herein, unless otherwise indicated, C₂₋₆alkenyl includes but is not limited to 1-propenyl, 2-propenyl, 1-butenyl, 2-butenyl and 3-butenyl.

As used herein, unless otherwise indicated, C₂₋₆alkynyl includes but is not limited to ethynyl, 1-propynyl, 2-propynyl, 1-butynyl, 2-butynyl and 3-butynyl.

As used herein, unless otherwise indicated, halo or halogen refers to fluorine, chlorine, bromine, or iodine;

As used herein, aryl includes to phenyl and naphthyl;

As used herein, aromatic or non-aromatic heterocyclic rings include but are not limited to N- or C-linked furyl, imidazolyl, oxazolyl, pyrrolidinyl, thiazolyl, thiophenyl, pyrrolyl, morpholinyl, piperidinyl, piperazinyl, pyrazinyl, pyridyl, pyrimidinyl, indanyl, indolyl, quinolinyl, isoquinolinyl, quinazolinyl, quinoxalinyl, benzo[b]thiophenyl, benzoxazolyl, or benzthiazolyl;

DCM refers to dichloromethane;

EtOAc refers to ethyl acetate;

EDC refers to 1-(3-dimethylaminopropyl)-3-ethylcarbodiimide;

EDTA refers to ethylenediaminetetraacetic acid;

HEPES refers to 4-(2-hydroxyethyl)-1-piperazine ethane sulfonic acid, monosodium salt, and TEA refers to triethylamine.

In processes described herein, where necessary, hydroxy, amino, or other reactive groups may be protected using a protecting group as described in the standard text “Protecting groups in Organic Synthesis”, 3^{rd} Edition (1999) by Greene and Wuts.

Unless otherwise stated, reactions are conducted under an inert atmosphere, preferably under a nitrogen atmosphere and are usually conducted at a pressure of about one to about three atmospheres, preferably at ambient pressure (about one atmosphere).

The compounds of the invention and intermediates may be isolated from their reaction mixtures by standard techniques.

Acid addition salts of the compounds of Formula I which may be mentioned include salts of mineral acids, for example the hydrochloride and hydrobromide salts; and salts formed with organic acids such as formate, acetate, maleate, benzoate, tartrate, and fumarate salts.

Acid addition salts of compounds of Formula I may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydroflran or diethyl ether, or a mixture of solvents, which may be removed in vacuum or by freeze drying. The reaction may be a metathetical process or it may be carried out on an ion exchange resin.

Certain compounds of Formula I may exist in tautomeric or enantiomeric forms, all of which are included within the scope of the invention. The various optical isomers may be isolated by separation of a racemic mixture of the compounds using conventional techniques, e.g. fractional crystallization, or chiral HPLC. Alternatively the individual enantiomers may be made by reaction of the appropriate optically active starting materials under reaction conditions which will not cause racemization.

### Synthesis and Schemes

Compounds of Formula 1 may be prepared generally by the process illustrated in Scheme 1.

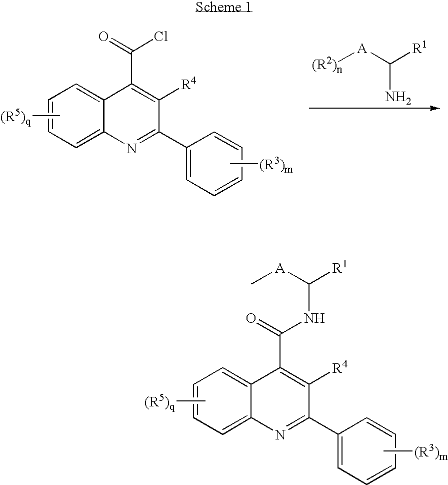

For example, reaction of 1-pyridin-3-yl-propylamine with 3-hydroxy-2-phenyl-quinoline-4-carbonyl chloride in ethyl acetate in the presence of a base such as triethyl amine will afford 3-hydroxy-2-phenyl-quinoline-4-carboxylic acid [1-(pyridin-3-yl)-propyl]-amide-3-Hydroxy-2-phenyl-quinoline-4-carbonyl chloride can be prepared by reacting 3-hydroxy-2-phenyl-quinoline-4-carboxylic acid with thionyl chloride in the presence of triethylamine in ethyl acetate. Alternatively, 3-hydroxy-2-phenyl-quinoline4-carboxylic acid [1-(pyridin-3-yl)-propyl]-amide may be prepared by reaction of 1-(pyridin-3-yl)-propylamine and 3-hydroxy-2-phenyl-quinoline-4-carboxylic acid in the presence of a suitable dehydrating reactant system such as dicyclohexylcarbodiimide and hydroxybenztriazole.

In a further aspect the invention relates to compounds described herein wherein one or more of the atoms is a radioisotope of the same element. In a particular form of this aspect of the invention the compound is labeled with tritium. Such radio-labeled compounds are synthesized either by incorporating radio-labeled starting materials or, in the case of tritium, exchange of hydrogen for tritium by known methods. Known methods include (1) electrophilic halogenation, followed by reduction of the halogen in the presence of a tritium source, for example, by hydrogenation with tritium gas in the presence of a palladium catalyst, or (2) exchange of hydrogen for tritium performed in the presence of tritium gas and a suitable organometallic (e.g. palladium) catalyst.

Compounds of the invention labeled with tritium are useful for the discovery of novel medicinal compounds which bind to and modulate the activity, by agonism, partial agonism, or antagonism, of an NK-3 receptor. Such tritium-labeled compounds may be used in assays that measure the displacement of such compounds to assess the binding of ligands that bind to NK-3 receptors.

In a further aspect the invention relates to compounds described herein additionally comprising one or more atoms of a radioisotope. In a particular form of this aspect of the invention the compound comprises a radioactive halogen. Such radio-labeled compounds are synthesized by incorporating radio-labeled starting materials by known methods. Particular embodiments of this aspect of the invention are those in which the radioisotope is selected from ¹⁸F, ¹²³I, ¹²⁵I, ¹³¹I, ⁷⁵Br, ⁷⁶Br, ⁷⁷Br or ⁸²Br. A most particular embodiment of this aspect of the invention is that in which the radioisotope is ¹⁸F. Such compounds comprising one or more atoms of a radioisotope are useful as positron emission tomography (PET) ligands and for other uses and techniques to determine the location of NK3 receptors. Therapeutic uses of compounds:

In another aspect the invention relates to compounds in accord with Formula I described herein and the use of such compounds in therapy and in compositions useful for therapy.

In another aspect the invention encompasses the use of compounds described herein for the therapy of diseases mediated through the action of NK-3 receptors. Such an aspect encompasses methods of treatment or prophylaxis of diseases or conditions in which modulation of the NK-3 receptor is beneficial which methods comprise administering a therapeutically-effective amount of an antagonistic compound of the invention to a subject suffering from said disease or condition.

One embodiment of this aspect of the invention is a method of treatment or prophylaxis of disorders, wherein the disorder is depression, anxiety, schizophrenia, cognitive disorders, psychoses, obesity, inflammatory diseases including irritable bowel syndrome and inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, or testicular cancer comprising administering a pharmacologically effective amount of a compound of Formula I to a patient in need thereof.

A further aspect of the invention is the use of a compound according to the invention, an enantiomer thereof or a pharmaceutically-acceptable salt thereof, for the treatment or prophylaxis of a disease or condition in which modulation of the NK-3 receptor is beneficial. Particular diseases and conditions that may be treated are depression, anxiety, schizophrenia, cognitive disorders, psychoses, obesity, inflammatory diseases including irritable bowel syndrome and inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, and testicular cancer. More particular embodiments encompass uses of a compound for treatment or prophylaxis of anxiety, depression, schizophrenia and obesity. A further aspect of the invention is the use of a compound according to the invention, an enantiomer thereof or a pharmaceutically-acceptable salt thereof, in the manufacture of a medicament for the treatment or prophylaxis of the diseases or conditions mentioned herein.

A particular embodiment of this aspect of the invention is the use of a compound of the invention in the manufacture of a medicament for treatment or prophylaxis of depression, anxiety, schizophrenia, cognitive disorders, psychoses, obesity, inflammatory diseases including irritable bowel syndrome and inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, and testicular cancer.

### Pharmaceutical Compositions

Compounds of the invention, enantiomers thereof, and pharmaceutically-acceptable. salts thereof, may be used on their own or in the form of appropriate medicinal preparations for enteral or parenteral administration. According to a further aspect of the invention, there is provided a pharmaceutical composition including preferably less than 80% and more preferably less than 50% by weight of a compound of the invention in admixture with an inert pharmaceutically-acceptable diluent, lubricant or carrier. Examples of diluents, lubricants and carriers are:
for tablets and dragees: lactose, starch, talc, stearic acid;
for capsules: tartaric acid or lactose;
for injectable solutions: water, alcohols, glycerin, vegetable oils;
for suppositories: natural or hardened oils or waxes.

There is also provided a process for the preparation of such a pharmaceutical composition which process comprises mixing or compounding the ingredients together and forming the mixed ingredients into tablets or suppositories, encapsulating the ingredients in capsules or dissolving the ingredients to form injectable solutions.

Pharmaceutically-acceptable derivatives include solvates and salts. For example, the compounds of the invention may form acid addition salts with acids, such as conventional pharmaceutically-acceptable acids including maleic, hydrochloric, hydrobromic, phosphoric, acetic, fumaric, salicylic, citric, lactic, mandelic, tartaric and methanesulfonic acids.

Acid addition salts of the compounds of Formula I which may be mentioned include salts of mineral acids, for example the hydrochloride and hydrobromide salts; and salts formed with organic acids such as formate, acetate, maleate, benzoate, tartrate, and fumarate salts. Acid addition salts of compounds of Formula I may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

For the uses, methods, medicaments and compositions mentioned herein the amount of compound used and the dosage administered will, of course, vary with the compound employed, the mode of administration and the treatment desired. However, in general, satisfactory results are obtained when the compounds of the invention are administered at a daily dosage of about 0.1 mg to about 20 mg/kg of animal body weight. Such doses may be given in divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400-mg of the compound admixed with a solid or liquid pharmaceutical carriers, lubricants and diluents.

Some compounds of the invention may exist in tautomeric, enantiomeric, stereoisomeric or geometric isomeric forms, all of which are included within the scope of the invention. The various optical isomers may be isolated by separation of a racemic mixture of the compounds using conventional techniques, e.g. fractional crystallization, or chiral HPLC. Alternatively the individual enantiomers may be made by reaction of the appropriate optically active starting materials under reaction conditions which will not cause racemization.

### Exemplary Compounds

Exemplary compounds of the invention may be prepared by processes analogous to that described in Scheme 1. Those skilled in the art will readily appreciate that many suitable amines and acid chlorides and carboxylic acids may be used to form compounds within the scope of the subject matter described herein as Formula I. The exemplary compounds and processes describe the invention by way of illustration and example for clarity of understanding. However to those skilled in the art, upon contemplation of the teaching of compounds, processes and methods of this invention, modifications and changes will be apparent that may be made thereto without departing from the spirit or scope of the invention.

### EXAMPLE 1

### 2-Phenyl-quinoline-4-carboxylic acid (1-pyridin-4-yl-propyl)-amide

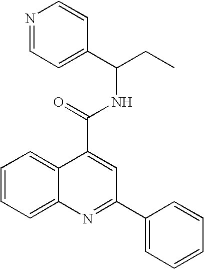

### EXAMPLE 2

### 3-Hydroxy-2-phenyl-quinoline-4-carboxylic Acid (1-pyridin-4-yl-propyl)-amide

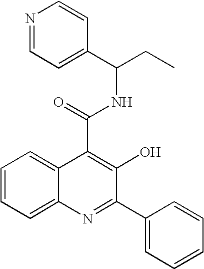

### EXAMPLE 3

### 3-Amino-2-phenyl-quinoline-4-carboxylic Acid (1-pyridin-4-yl-propyl)-amide

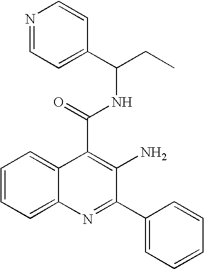

### EXAMPLE 4

### 3-Methyl-2-phenyl-quinoline-4-carboxylic Acid (1-pyridin-4-yl-propyl)-amide

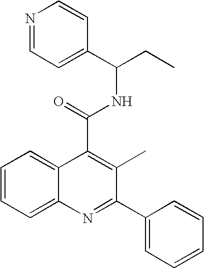

### EXAMPLE 5

### 3-Methoxy-2-phenyl-quinoline-4-carboxylic Acid (1-pyridin-4-yl-propyl)-amide

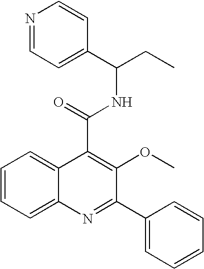

### EXAMPLE 6

### 2-Phenyl-quinoline-4-carboxylic Acid (1-pyridin-4-yl-ethyl)-amide

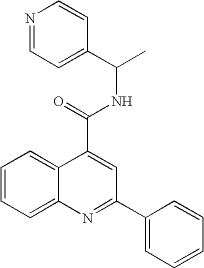

### EXAMPLE 7

### 3-Hydroxy-2-phenyl-quinoline-4-carboxylic Acid (1-pyridin-4-yl-ethyl)-amide

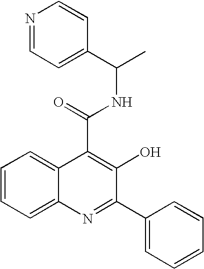

### EXAMPLE 8

### 3-Amino-2-phenyl-quinoline-4-carboxylic Acid (1-pyridin-4-yl-ethyl)-amide

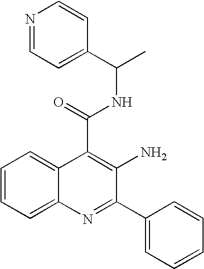

### EXAMPLE 9

### 3-Methyl-2-phenyl-quinoline-4-carboxylic Acid (1-pyridin-4-yl-ethyl)-amide

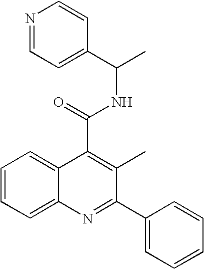

### EXAMPLE 10

### 3-Methoxy-2-phenyl-quinoline-4-carboxylic Acid (1-pyridin-4-yl-propyl)-amide

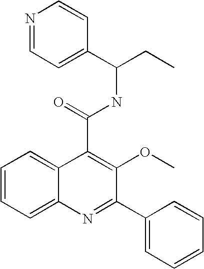

### EXAMPLE 11

### [(2-Phenyl-quinoline-4-carbonyl)-amino]-pyridin-4-yl-acetic Acid Methyl Ester

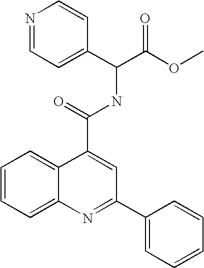

### EXAMPLE 12

### [(3-Hydroxy-2-phenyl-quinoline-4-carbonyl)-amino]-(pyridin-4-yl)-acetic Acid Methyl Ester

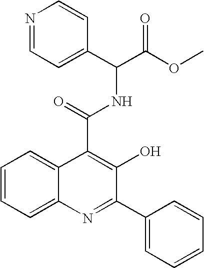

### EXAMPLE 13

### [(3-Amino-2-phenyl-quinoline-4-carbonyl)-amino]-(pyridin-4-yl)-acetic Acid Methyl Ester

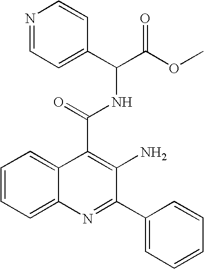

### EXAMPLE 14

### [(3-Methyl-2-phenyl-quinoline-4-carbonyl)-amino]-(pyridin-4-yl)-acetic Acid Methyl Ester

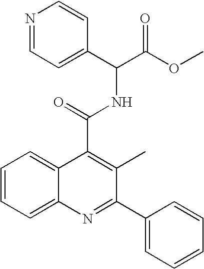

### EXAMPLE 15

### [(3-Methoxy-2-phenyl-quinoline-4-carbonyl)-amino]-(pyridin-4-yl)-acetic Acid Methyl Ester

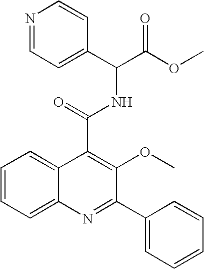

### EXAMPLE 16

### 2-Phenyl-quinoline-4-carboxylic Acid (1-pyridin-3-yl-propyl)-amide

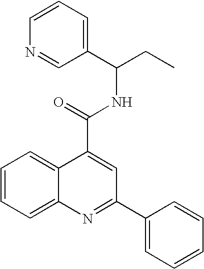

### EXAMPLE 17

### 3-Hydroxy-2-phenyl-quinollne-4-carboxylic Acid (1-pyridin-3-yl-propyl)-amide

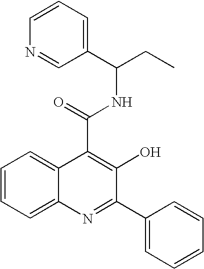

(a) 1-pyridin-3-ylpropan-1-amine:

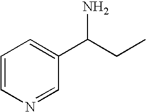

3-Propanoylpyridine (1.0 g, 7.41 mmol), formamide (1.5 mL, 37.1 mmol), and formic acid (1.1 mL, 29.6 mnmol) were combined in a reaction tube and heated at 160° C. (external temperature), with stirring, overnight. It was cooled, diluted with water and saturated aqueous Na₂CO₃ (approx. 50:50) and extracted with EtOAc. The aqueous phase was then saturated with NaCl and extracted with CH₂Cl₂, then EtOAc. The organic phases were dried over Na₂SO₄, filtered and concentrated under reduced pressure. The residue was transferred to a reaction tube, dissolved in 2 N HCl, and heated at 100° C. overnight. It was then concentrated under reduced pressure, dissolved/suspended in EtOH and CH₃CN, concentrated under reduced pressure, suspended in CH₃CN, and again concentrated under reduced pressure to give the desired product (1.2 g, 80% yield) as a solid (bis HCl salt). ¹H NMR (300 MHz, MeOD) δ 9.10 (s, 1H), 8.98 (d, J=5.6 Hz, 1H), 8.82 (d, J=6.7 Hz, 1H), 8.28-8.19 (m, 1H), 4.62 (t, J=7.6 Hz, 1H), 2.29-2.04 (m, 2H), 0.99 (t, J=7.4 Hz, 3H); m/z 137 (MH⁺).

To prepare the title compound a stirring solution of 3-hydroxy-2-phenylquinoline-4-carboxylic acid (150 mg, 0.57 mmol) and triethylamine (0.20 mL, 1.43 mmol) in EtOAc (6 mL) at −5 ° C. was added thionyl chloride (0.049 mL, 0.68 mmol). The cooling bath was removed and reaction allowed to stir 50 min., then a solution of 1-pyridin-3-ylpropan-1-amine (142 mg of the bis HCl salt, 0.68 mmol) and triethylamine (0.25 mL, 1.8 mmol) in EtOAc (1 mL) and NMP (1 mL) was added. The reaction was allowed to stir for 10 min., then heated at 80 ° C. (external temperature) for 1 h. It was cooled, diluted with EtOAc, and washed with a small quantity of aqueous 0.1 N NaOH (with some NaCl added to reduce emulsion formation). The organics were dried over Na₂SO₄, filtered, and concentrated under reduced pressure. The residue was purified by silica gel chromatography (EtOAc/CH₂Cl₂) to give the desired product (110 mg, 50% yield) as a yellow foam solid. ¹H NMR (300 MHz, CDCl3) δ 11.06 (s, 1H), 8.70 (s, 1H), 8.56 (s, 1H), 8.20-8.11 (m, 1H), 8.09-7.97 (m, 3H), 7.70 (d, J=7.7 Hz, 1H), 7.61- 7.43 (m, 5H), 7.37-7.28 (m, 1H), 6.75 (d, J=7.7 Hz, 1H), 5.31-5.19 (m, 1H), 2.15-1.95 (m, 2H), 1.06 (t, J=7.4 Hz, 3H); HRMS m/z 384.1668, calcd. 384.1712.

### EXAMPLE 18

### 3-Amino-2-phenyl-quinoline-4-carboxylic Acid (1-pyridin-3-yl-propyl)-amide

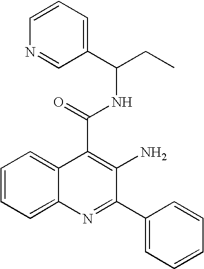

### EXAMPLE 19

### 3-Methyl-2-phenyl-quinoline-4-carboxylic Acid (1-pyridin-3-yl-propyl)-amide

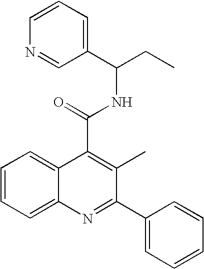

### EXAMPLE 20

### 3-Methoxy-2-phenyl-quinoline-4-carboxylic Acid (1-pyridin-3-yl-propyl)-amide

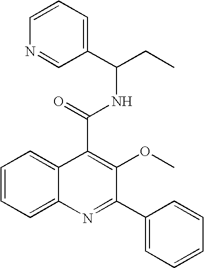

### EXAMPLE 21

### 2-Phenyl-quinoline-4-carboxylic Acid (1-pyridin-3-yl-ethyl)-amide

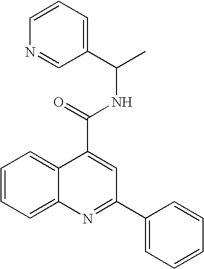

### EXAMPLE 22

### 3-Hydroxy-2-phenyl-quinoline-4-carboxylic Acid (1-pyridin-3-yl-ethyl)-amide

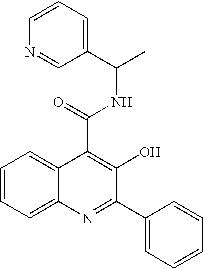

### EXAMPLE 23

### 3-Amino-2-phenyl-quinoline-4-carboxylic Acid (1-pyridin-3-yl-ethyl)-amide

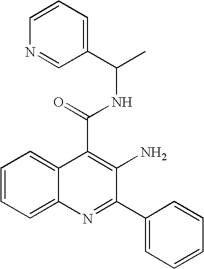

### EXAMPLE 24

### 3-Methyl-2-phenyl-quinoline-4-carboxylic Acid (1-pyridin-3-yl-ethyl)-amide

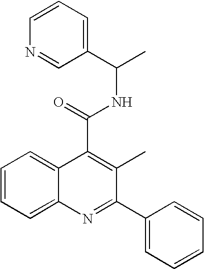

### EXAMPLE 25

### 3-Methoxy-2-phenyl-quinoline-4-carboxylic Acid (1-pyridin-3-yl-propyl)-amide

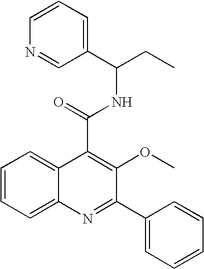

### EXAMPLE 26

### [(2-Phenyl-quinoline-4-carbonyl)-amino]-pyridin-3-yl-acetic Acid Methyl ester

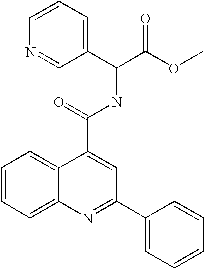

### EXAMPLE 27

### [(3-Hydroxy-2-phenyl-quinoline-4-carbonyl)-amino]-(pyridin-3-yl)-acetic Acid Methyl Ester

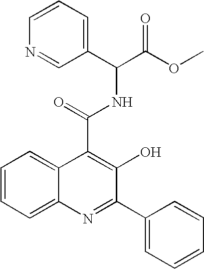

### EXAMPLE 28

### [(3-Amino-2-phenyl-quinoline-4-carbonyl)-amino]-(pyridin-3-yl)-acetic Acid Methyl Ester

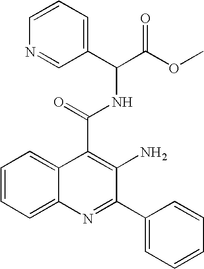

### EXAMPLE 29

### [(3-Methyl-2-phenyl-quinoline-4-carbonyl)-amino]-(pyridin-3-yl)-acetic Acid Methyl Ester

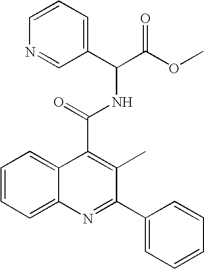

### EXAMPLE 30

### [(3-Methoxy-2-phenyl-quinoline-4-carbonyl)-amino]-(pyridin-3-yl)-acetic Acid Methyl Ester

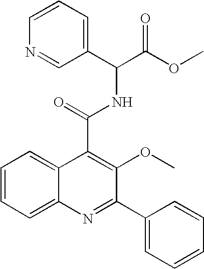

### EXAMPLE 31

### 2-Phenyl-quinoline-4-carboxylic Acid (1-pyridin-2-yl-propyl)-amide

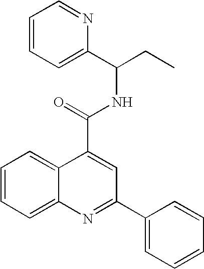

### EXAMPLE 32

### 3-Hydroxy-2-phenyl-quinoline-4-carboxylic Acid (1-pyridin-2-yl-propyl)-amide

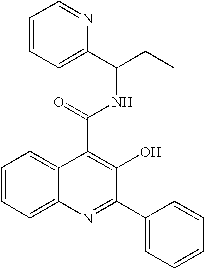

### EXAMPLE 33

### 3-Amino-2-phenyl-quinoline-4-carboxylic Acid (1-pyridin-2-yl-propyl)-amide

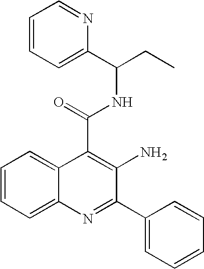

### EXAMPLE 34

### 3-Methyl-2-phenyl-quinoline-4-carboxylic Acid (1-pyridin-2-yl-propyl)-amide

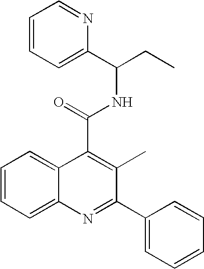

### EXAMPLE 35

### 3-Methoxy-2-phenyl-quinoline-4-carboxylic Acid (1-pyridin-2-yl-propyl)-amide

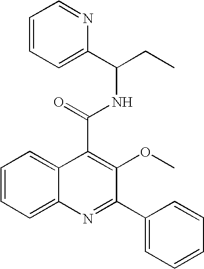

### EXAMPLE 36

### 2-Phenyl-quinoline-4-carboxylic Acid (1-pyridin-2-yl-ethyl)-amide

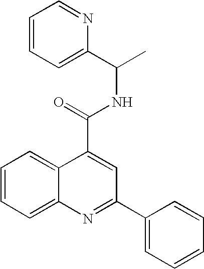

### EXAMPLE 37

### 3-Hydroxy-2-phenyl-quinoline-4-carboxylic Acid (1-pyridin-2-yl-ethyl)-amide

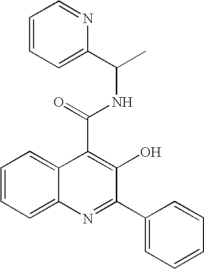

### EXAMPLE 38

### 3-Amino-2-phenyl-quinoline-4-carboxylic Acid (1-pyridin-2-yl-ethyl)-amide

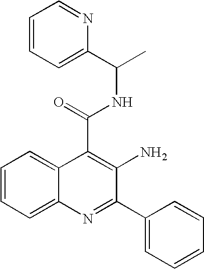

### EXAMPLE 39

### 3-Methyl-2-phenyl-quinoline-4-carboxylic Acid (1-pyridin-2-yl-ethyl)-amide

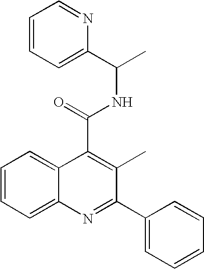

### EXAMPLE 40

### 3-Methoxy-2-phenyl-quinoline-4-carboxylic Acid (1-pyridin-2-yl-propyl)-amide

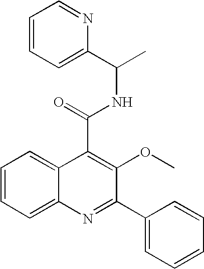

### EXAMPLE 41

### [(2-Phenyl-quinoline-4-carbonyl)-amino]-pyridin-2-yl-acetic Acid Methyl Ester

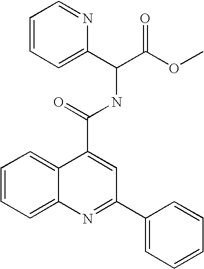

### EXAMPLE 42

### [(3-Hydroxy-2-phenyl-quinoline-4-carbonyl)-amino]-(pyridin-2-yl)-acetic Acid Methyl Ester

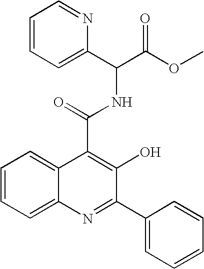

### EXAMPLE 43

### [(3-Amino-2-phenyl-quinoline-4-carbonyl)-amino]-(pyridin-2-yl)-acetic Acid Methyl Ester

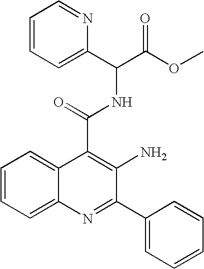

### EXAMPLE 44

### [(3-Methyl-2-phenyl-quinoline-4-carbonyl)-amino]-(pyridin-2-yl)-acetic Acid Methyl Ester

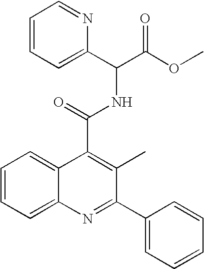

### EXAMPLE 45

### [(3-Methoxy-2-phenyl-quinoline-4-carbonyl)-amino]-(pyridin-2-yl)-acetic Acid Methyl Ester

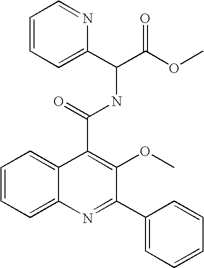

### Biological Tests

### NK-3 Receptor Binding Activity:

Generally, NK-3r binding activity may be assessed using assays performed as described in Krause et al (Proc. Natl. Acad. Sci. USA 94: 310-315, 1997). NK-3r complementary DNA is cloned from human hypothalamic RNA using standard procedures. The receptor cDNA is inserted into a suitable expression vector transfected into a Chinese hamster ovary cell line, and a stably-expressing clonal cell line may be isolated, characterized and used for experiments.

Cells may be grown in tissue culture medium by techniques known to those of skill in the art and recovered by low speed centrifugation. Cell pellets may be homogenized, total cellular membranes isolated by high speed centrifugation and resuspended in buffered saline. Generally, receptor binding assays may be performed by incubating suitable amounts of purified membrane preparations with ¹²⁵I-methylPhe7-neurokinin B, in the presence or absence of test compounds. Membrane proteins may be harvested by rapid filtration and radioactivity may be quantitated in a β-plate scintillation counter. Nonspecific binding may be distinguished from specific binding by use of suitable controls and the affinity of compounds for the expressed receptor may be determined by using different concentrations of compounds.

### Preparation of Membranes From CHO Cells Transfected With Cloned NK-3 Receptors:

A human NK-3 receptor gene was cloned using methods similar to those described for other human NK receptors (Aharony et al., Mol. Pharmacol. 45:9-19, 1994; Caccese et al., Neuropeptides 33, 239-243, 1999). The DNA sequence of the cloned NK-3 receptor differed from the published sequence (Buell et al., FEBS Letts. 299, 90-95, 1992; Huang et al., Biochem. Biophys. Res. Commun. 184, 966-972, 1992) having a silent single T>C base change at nucleotide 1320 of the coding sequence. Since the change is silent, the cloned gene provides a primary amino acid sequence for the encoded NK-3 receptor protein identical to the published sequence. The receptor cDNA was used to transfect CHO-K1 cells using standard methods and a clone stably-expressing the receptor was isolated and characterized. Plasma membranes from these cells were prepared as published (Aharony et al., 1994).

Cells were harvested and centrifuged to remove medium. The pelleted cells were homogenized (Brinkman Polytron, three 15 sec bursts on ice) in a buffer consisting of 50 mM Tris-HCl (pH 7.4), 120 mM NaCl, 5 mM KCl, 10 mM EDTA and protease inhibitors (0.1 mg/ml soybean trypsin inhibitor, and 1 mM iodoacetamide). The homogenate was centrifuged at 1000×g for 10 min at 4 ° C. to remove cell debris. Pellets were washed once with homogenizing buffer. Supernatants were combined and centrifuged at 40,000×g for 20 min at 4° C. The membrane-containing pellet was homogenized with a Polytron as before. The suspension was centrifuged at 40,000×g for 20 min at 4° C. and resuspended in buffer (20 mM HEPES, pH 7.4 containing 3 mM MgCl₂, 30 mM KCl, and 100 μM thiorphan) and the protein concentration determined. The membrane suspension was then diluted to 3 mg/ml with buffer containing 0.02% BSA, and flash frozen. Samples were stored at −80 ° C. until used.

### Assay for NK-3 Receptor Binding Activity:

A receptor binding assay method with [¹²⁵]-MePhe7-NKB was modified from that described by Aharony et al., J. Pharmacol. Exper. Ther., 274:1216-1221, 1995.

Competition experiments were carried out in 0.2 mL assay buffer (50 mM Tris-HCl, 4 mM MnCl₂, 10 μM thiorphan, pH 7.4) containing membranes (2 μg protein/reaction), tested competitors, and [¹²⁵I]-MePhe7NKB (0.2 nM). Unlabeled homologue ligand (0.5 μM) was used to define nonspecific binding. Incubations were carried out at 25° C. for 90 min. Receptor-bound ligand was isolated by vacuum filtration in a Packard Harvester onto GF/C plates presoaked in 0.5% BSA. Plates were washed with 0.02 M Tris, pH 7.4. Computation of equilibrium binding constants (K_{D} and Ki), receptor density (Bmax), and statistical analysis was carried out as published previously (Aharony et al., 1995) using GraphPad Prism or IDBS XLfit software.

### NK-3 Functional Activity:

Generally, NK-3 functional activity may be assessed by using calcium mobilization assays in stable NK-3r-expressing cell lines. Calcium mobilization induced by the methylPhe7-neurokinin B agonist may be monitored using a FLIPR (Molecular Devices) instrument in the manner described by the manufacturer. Agonists may be added to the cells and fluorescence responses continuously recorded for up to 5 min. The actions of antagonists may be assessed by preincubating cells prior to administration of the methylPhe7-neurokinin B agonist. The action of agonists may be assessed by observing their intrinsic activity in such a system.

### Assay for NK-3 Functional Activity:

NK-3 receptor expressing CHO cells were maintained in growth media (Ham's F12 medium, 10% FBS, 2 mM L-glutamine, and 50 mg/mL Hygromycin B). One day prior to the assay cells were dispensed into 384-well plates in Ultraculture media (Cambrex Bio Science) with 2 mM L-glutamine to achieve 70-90% confluency. To quantify NK-3 receptor-induced calcium mobilization, cells were first washed with assay buffer consisting of Hanks Balanced Salt Solution, 15 mM HEPES, and 2.5 mM probenecid, pH 7.4. The cells were then loaded with Fluo4/AM dye (4.4 μM) in assay buffer. Cells were incubated for one hour and then washed with assay buffer, exposed to 0.02-300 nM senktide and the fluorescence response recorded using a FLIPR instrument (Molecular Devices Corporation). To quantify antagonism of the agonist response, cells were preincubated with varying concentrations of test compound for 2-20 min and then exposed to 2 nM senktide, a concentration that alone elicits about an 70% maximal calcium response. The resulting data was analyzed using XLfit software (IDBS manufacturer) to determine EC50 and IC50 values.

1. A compound in accord with Formula I. 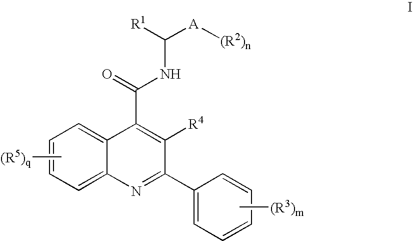
wherein:
R¹ is selected from H, C₁₋₄alkyl-, C₃₋₆cycloalkyl- and C₁₋₄alkylOC(O)-;
A is pyridyl;
R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;
n is 1, 2 or 3;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1, 2 or 3;
R⁴ is selected from H, —OH, —NH₂, —OSO₂R⁶, C₁₋₄alkyl-, C₁₋₄alkoxy-, C₁₋₆alkoxyC₁₋₆alkyl-, and E-(CH₂)ₚ-, where E is selected from —NR⁶R⁷, —SOC₁₋₆alkyl, —SO₂C₁₋₆alkyl, —NR⁶SO₂R⁷, —SR⁶, N⁺(O⁻)R⁶R⁷, aryl and an N- or C-linked 5- or 6-membered aromatic or non-aromatic heterocyclic ring having 1, 2, 3 or 4 nitrogen atoms or an N-oxide thereof, and p is 0, 1, 2, 3, 4 or 5;
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
and,
when R⁴ is E-(CH₂)ₚ- and said E thereof is an N or C linked 5- or 6-membered aromatic or non-aromatic heterocyclic ring or an N-oxide thereof, said E is unsubstituted or has 1, 2 or 3 substituents independently selected from —OH, ═O, —NH₂, —CN, halogen, C₁₋₄alkyl-, C₁₋₄alkoxy-, C₁₋₄alkyl-CO—, —NR⁶R⁷, aryl and a 5- or 6-membered aromatic or non-aromatic heterocyclic ring having 1, 2, 3 or 4 nitrogen atoms;
and,
when R¹, R², R³ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen;
or a stereoisomer, enantiomer, in vivo-hydrolysable precursor or pharmaceutically-acceptable salt thereof.

2. A compound according to claim 1, wherein:
A is selected from pyrid-2-yl, pyrid-3-yl and pyrid-4-yl;
R¹ is selected from C₁₋₄alkyl-, C₃₋₆cycloalkyl- and C₁₋₄alkylOC(O)-;
R² is selected from H, halogen and unsubstituted C₁₋₆alkoxy-;
R³ is H or halogen;
n and m are both 1, and
when R¹ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN and halogen;
or a stereoisomer, enantiomer, in vivo-hydrolysable precursor or pharmaceutically-acceptable salt thereof.

3. A compound according to claim 1, wherein:
A is selected from pyrid-2-yl, pyrid-3-yl and pyrid-4-yl;
R¹ is selected from C₁₋₄alkyl- and C₃₋₆cycloalkyl-;
R² is selected from H, halogen and unsubstituted C₁₋₆alkoxy-;
R³ is H or halogen;
n and m are both 1;
R⁴ is selected from H, —OH, —NH₂, C₁₋₄alkyl-, C₁₋₄alkoxy- and E-(CH₂)ₚ-, where E is a substituted or unsubstituted N-linked 5- or 6-membered aromatic or non-aromatic heterocyclic ring having 1, 2, 3 or 4 nitrogen atoms, and
R⁵ is H;
or a stereoisomer, enantiomer, in vivo-hydrolysable precursor or pharmaceutically-acceptable salt thereof.

4. A compound according to claim 1, wherein:
A is selected from pyrid-2-yl, pyrid-3-yl and pyrid-4-yl;
R¹ is ethyl or cyclopropyl;
R² is selected from H, F and —OCH₃;
R³ is H or F;
n, m and q are each 1;
R⁴ is selected from H, —OH, —CH₃, —OCH₃, and NH₂, and
R⁵ at each occurrence is independently selected from H, —OH and halogen;
or a stereoisomer, enantiomer, in vivo-hydrolysable precursor or pharmaceutically-acceptable salt thereof.

5. A compound according to claim 1, in accord with Formula II 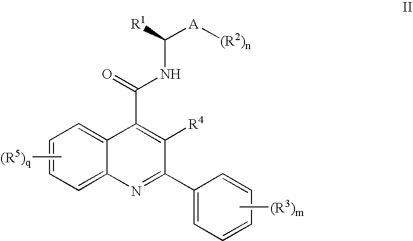
wherein R¹, A, R², n, R³, m, R⁴, R⁵ and q are as defined for Formula I;
or a stereoisomer, enantiomer, in vivo-hydrolysable precursor or pharmaceutically-acceptable salt thereof.

6. A compound according to claim 1, selected from:
2-Phenyl-quinoline-4-carboxylic acid (1-pyridin-4-yl-propyl)-amide;
3-Hydroxy-2-phenyl-quinoline-4-carboxylic acid (1-pyridin-4-yl-propyl)-amide;
3-Amino-2-phenyl-quinoline-4-carboxylic acid (1-pyridin-4-yl-propyl)-amide;
3-Methyl-2-phenyl-quinoline-4-carboxylic acid (1-pyridin-4-yl-propyl)-amide;
3-Methoxy-2-phenyl-quinoline-4-carboxylic acid (1-pyridin-4-yl-propyl)-amide;
2-Phenyl-quinoline-4-carboxylic acid (1-pyridin-4-yl-ethyl)-amide;
3-Hydroxy-2-phenyl-quinoline-4-carboxylic acid (1-pyridin-4-yl-ethyl)-amide;
3-Amino-2-phenyl-quinoline-4-carboxylic acid (1-pyridin-4-yl-ethyl)-amide;
3-Methyl-2-phenyl-quinoline-4-carboxylic acid (1-pyridin-4-yl-ethyl)-amide;
3-Methoxy-2-phenyl-quinoline-4-carboxylic acid (1-pyridin-4-yl-propyl)-amide;
[(2-Phenyl-quinoline-4-carbonyl)-amino]-pyridin-4-yl-acetic acid methyl ester;
[(3-Hydroxy-2-phenyl-quinoline-4-carbonyl)-amino]-(pyridin-4-yl)-acetic acid methyl ester;
[(3-Amino-2-phenyl-quinoline-4-carbonyl)-amino]-(pyridin-4-yl)-acetic acid methyl ester;
[(3-Methyl-2-phenyl-quinoline-4-carbonyl)-amino]-(pyridin-4-yl)-acetic acid methyl ester;
[(3-Methoxy-2-phenyl-quinoline-4-carbonyl)-amino]-(pyridin-4-yl)-acetic acid methyl ester;
2-Phenyl-quinoline-4-carboxylic acid (1-pyridin-3-yl-propyl)-amide;
3-Hydroxy-2-phenyl-quinoline-4-carboxylic acid (1-pyridin-3-yl-propyl)-amide;
3-Amino-2-phenyl-quinoline-4-carboxylic acid (1-pyridin-3-yl-propyl)-amide;
3-Methyl-2-phenyl-quinoline-4-carboxylic acid (1-pyridin-3-yl-propyl)-amide;
3-Methoxy-2-phenyl-quinoline-4-carboxylic acid (1-pyridin-3-yl-propyl)-amide;
2-Phenyl-quinoline-4-carboxylic acid (1-pyridin-3-yl-ethyl)-amide;
3-Hydroxy-2-phenyl-quinoline-4-carboxylic acid (1-pyridin-3-yl-ethyl)-amide;
3-Amino-2-phenyl-quinoline-4-carboxylic acid (1-pyridin-3-yl-ethyl)-amide;
3-Methyl-2-phenyl-quinoline-4-carboxylic acid (1-pyridin-3-yl-ethyl)-amide;
3-Methoxy-2-phenyl-quinoline-4-carboxylic acid (1-pyridin-3-yl-propyl)-amide;
[(2-Phenyl-quinoline-4-carbonyl)-amino]-pyridin-3-yl-acetic acid methyl ester;
[(3-Hydroxy-2-phenyl-quinoline-4-carbonyl)-amino]-(pyridin-3-yl)-acetic acid methyl ester;
[(3-Amino-2-phenyl-quinoline-4-carbonyl)-amino]-(pyridin-3-yl)-acetic acid methyl ester;
[(3-Methyl-2-phenyl-quinoline-4-carbonyl)-amino]-(pyridin-3-yl)-acetic acid methyl ester;
[(3-Methoxy-2-phenyl-quinoline-4-carbonyl)-amino]-pyridin-3-yl)-acetic acid methyl ester;
2-Phenyl-quinoline-4-carboxylic acid (1-pyridin-2-yl-propyl)-amide;
3-Hydroxy-2-phenyl-quinoline-4-carboxylic acid (1-pyridin-2-yl-propyl)-amide;
3-Amino-2-phenyl-quinoline-4-carboxylic acid (1-pyridin-2-yl-propyl)-amide;
3-Methyl-2-phenyl-quinoline-4-carboxylic acid (1-pyridin-2-yl-propyl)-amide;
3-Methoxy-2-phenyl-quinoline-4-carboxylic acid (1-pyridin-2-yl-propyl)-amide;
2-Phenyl-quinoline-4-carboxylic acid (1-pyridin-2-yl-ethyl)-amide;
3-Hydroxy-2-phenyl-quinoline-4-carboxylic acid (1-pyridin-2-yl-ethyl)-amide;
3-Amino-2-phenyl-quinoline-4-carboxylic acid (1-pyridin-2-yl-ethyl)-amide;
3-Methyl-2-phenyl-quinoline-4-carboxylic acid (1-pyridin-2-yl-ethyl)-amide;
3-Methoxy-2-phenyl-quinoline-4-carboxylic acid (1-pyridin-2-yl-propyl)-amide;
[(2-Phenyl-quinoline-4-carbonyl)-amino]-pyridin-2-yl-acetic acid methyl ester;
[(3-Hydroxy-2-phenyl-quinoline-4-carbonyl)-amino]-(pyridin-2-yl)-acetic acid methyl ester;
[(3-Amino-2-phenyl-quinoline-4-carbonyl)-amino]-(pyridin-2-yl)-acetic acid methyl ester;
[(3-Methyl-2-phenyl-quinoline-4-carbonyl)-amino]-(pyridin-2-yl)-acetic acid methyl ester, and
[(3-Methoxy-2-phenyl-quinoline-4-carbonyl)-amino]-(pyridin-2-yl)-acetic acid methyl ester.

7. A process for preparing a compound of Formula I, 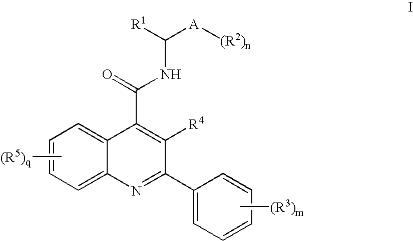
wherein:
R¹ is selected from H, C₁₋₄alkyl-, C₃₋₆cycloalkyl- and C₁₋₄alkylOC(O)-;
A is pyridyl;
R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;
n is 1, 2 or 3;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1, 2 or 3;
R⁴ is selected from H, —OH, —NH₂, —OSO₂R⁶, C₁₋₄alkyl-, C₁₋₄alkoxy-, C₁₋₆alkoxyC₁₋₆alkyl-, and E-(CH₂)ₚ-, where E is selected from —NRE⁶R⁷, —SOC₁₋₆alkyl, —SO₂C₁₋₆alkyl, —NR⁶SO₂R⁷, —SR⁶, N⁺(O⁻)R⁶R⁷, aryl and an N- or C-linked 5- or 6-membered aromatic or non-aromatic heterocyclic ring having 1, 2, 3 or 4 nitrogen atoms or an N-oxide thereof, and p is 0, 1, 2, 3, 4 or 5;
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
and,
when R⁴ is E-(CH₂)ₚ- and said E thereof is an N or C linked 5- or 6-membered aromatic or non-aromatic heterocyclic ring or an N-oxide thereof, said E is unsubstituted or has 1, 2 or 3 substituents independently selected from —OH, ═O, —NH₂, —CN, halogen, C₁₋₄alkyl-, C₁₋₄alkoxy-, C₁₋₄alkyl-CO—, —NR⁶R⁷, aryl and a 5- or 6-membered aromatic or non-aromatic heterocyclic ring having 1, 2, 3 or 4 nitrogen atoms; and,
when R¹, R², R³ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen; said process comprising:
preparing a 2-phenyl-quinolinyl-4-carbonyl chloride by reacting a 2-phenyl-quinolinyl-4-carboxylic acid with thionyl chloride in the presence of triethylamine in ethyl acetate;
reacting said 2-phenyl-quinoline-4-carbonyl chloride with a pyridinyl-propylamine in ethyl acetate in the presence of a base to yield a 2-phenyl-quinoline-4-carboxylic acid pyridinyl-propyl-amide of Formula I.

8. A process for preparing a compound of Formula I, 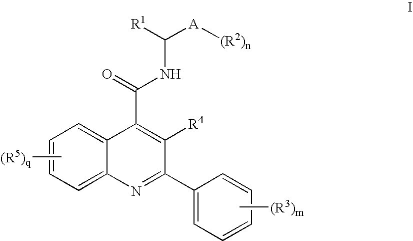
wherein:
R¹ is selected from H, C₁₋₄alkyl-, C₃₋₆cycloalkyl- and C₁₋₄alkylOC(O)-;
A is pyridyl;
R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;
n is 1, 2 or 3;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1, 2 or 3;
R⁴ is selected from H, —OH, —NH₂, —OSO₂R⁶, C₁₋₄alkyl-, C₁₋₄alkoxy-, C₁₋₆alkoxyC₁₋₆alkyl-, and E-(CH₂)ₚ-, where E is selected from —NR⁶R⁷, —SOC₁₋₆alkyl, —SO₂C₁₋₆alkyl, —NR⁶SO₂R⁷, —SR⁶, N^{+(O}⁻)R⁶R⁷, aryl and an N- or C-linked 5- or 6-membered aromatic or non-aromatic heterocyclic ring having 1, 2, 3 or 4 nitrogen atoms or an N-oxide thereof, and p is 0, 1, 2, 3, 4 or 5;
R⁵ at each occurrence is independently selected from H, —OH, —CN, halogen, —R⁶, —OR⁶—NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
and,
when R⁴ is E-(CH₂)ₚ- and said E thereof is an N or C linked 5- or 6-membered aromatic or non-aromatic heterocyclic ring or an N-oxide thereof, said E is unsubstituted or has 1, 2 or 3 substituents independently selected from —OH, ═O, —NH₂, —CN, halogen, C₁₋₄alkyl-, C₁₋₄alkoxy-, C₁₋₄alkyl-CO—, —NR₆R⁷, aryl and a 5- or 6-membered aromatic or non-aromatic heterocyclic ring having 1, 2, 3 or 4 nitrogen atoms;
and,
when R¹, R², R³ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen;
said process comprising:
reacting an amine of the following formula: 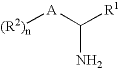
with a 2-phenyl-quinolinyl-4-carbonyl chloride of the following formula 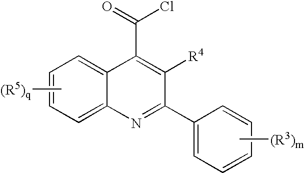
to afford a compound of Formula I.

9. A method of treatment or prophylaxis of a disease or condition in which modulation of the NK3 receptor is beneficial which method comprises administering to a subject suffering from said disease or condition a therapeutically-effective amount of a compound in accord with Formula I: 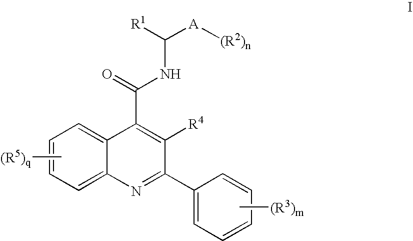
wherein:
R¹ is selected from H, C₁₋₄alkyl-, C₃₋₆cycloalkyl- and C₁₋₄alkylOC(O)-;
A is pyridyl;
R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;
n is 1, 2 or 3;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1 ,2 or 3;
R⁴ is selected from H, —OH, —NH₂, —OSO₂R^{b}, C₁₋₄alkyl-, C₁₋₄alkoxy-, C₁₋₆alkoxyC₁₋₆alkyl-, and E-(CH₂)ₚ-, where E is selected from —NR⁶R⁷, —SOC₁₋₆alkyl, —SO₂C₁₋₆alkyl, —NR⁶So₂R⁷, —SR⁶, N⁺(O⁻)R⁶R⁷, aryl and an N- or C-linked 5- or 6-membered aromatic or non-aromatic heterocyclic ring having 1, 2, 3 or 4 nitrogen atoms or an N-oxide thereof, and p is 0, 1, 2, 3, 4 or 5;
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
and,
when R⁴ is E-(CH₂)ₚ- and said E thereof is an N or C linked 5- or 6-membered aromatic or non-aromatic heterocyclic ring or an N-oxide thereof, said E is unsubstituted or has 1, 2 or 3 substituents independently selected from —OH, ═O, -NI12, —CN, halogen, C₁₋₄alkyl-, C₁₋₄alkoxy-, C₁₋₄alkyl-CO—, —NR⁶R7, aryl and a 5- or 6-membered aromatic or non- aromatic heterocyclic ring having 1, 2, 3 or 4 nitrogen atoms; and,
when R¹, R², R³ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen;
or a stereoisomer, enantiomer, in vivo-hydrolysable precursor or pharmaceutically-acceptable salt thereof.

10. The method of claim 9, wherein said disease or condition is selected from depression, anxiety, schizophrenia, cognitive disorders, psychoses, obesity, inflammatory diseases, irritable bowel syndrome, inflammatory bowel disorder, emesis, pre-eclampsia, chronic obstructive pulmonary disease, disorders associated with excessive gonadotrophins and/or androgens including dysmenorrhea, benign prostatic hypberplasia, prostatic cancer, and testicular cancer.

11. A pharmaceutical composition comprising a pharmaceutically-acceptable diluent, lubricant or carrier and a compound in accord with Formula I: 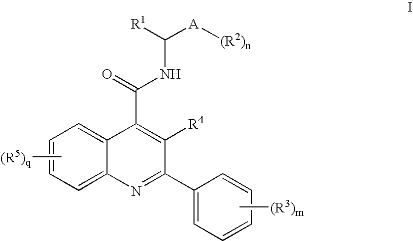
wherein:
R¹ is selected from H, C₁₋₄alkyl-, C₃₋₆cycloalkyl- and C₁₋₄alkylOC(O)-;
A is pyridyl;
R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;
n is 1, 2 or 3;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1, 2 or 3;
R⁴ is selected from H, —OH, —NH₂, —OSO₂R⁶, C₁₋₄alkyl-, C_{1- 4}alkoxy-, C₁₋₆alkoxyC₁₋₆alkyl-, and E-(CH₂)ₚ-, where E is selected from —NR⁶R⁷, —SOC₁₋₆alkyl, —SO₂C₁₋₆alkyl, —NR⁶SO₂R⁷, —SR⁶, N⁺(O⁻)R⁶R⁷, aryl and an N- or C-linked 5- or 6-membered aromatic or non-aromatic heterocyclic ring having 1, 2, 3 or 4 nitrogen atoms or an N-oxide thereof, and p is 0, 1, 2, 3, 4 or 5;
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁰ and R¹ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
and,
when R⁴ is E-(CH₂)ₚ- and said E thereof is an N or C linked 5- or 6-membered aromatic or non-aromatic heterocyclic ring or an N-oxide thereof, said E is unsubstituted or has 1, 2 or 3 substituents independently selected from —OH, ═O, —NH₂, —CN, halogen, C₁₋₄alkyl-, C₁₋₄alkoxy-, C₁₋₄alkyl-CO—, —NR⁶R⁷, aryl and a 5- or 6-membered aromatic or non-aromatic heterocyclic ring having 1, 2, 3 or 4 nitrogen atoms;
and,
when R¹, R², R³ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen;
or a stereoisomer, enantiomer, in vivo-hydrolysable precursor or pharmaceutically-acceptable salt thereof.

12. A method of treatment or prophylaxis of a disease or condition in which modulation of the NK3 receptor is beneficial which method comprises administering a therapeutically-effective amount of a pharmaceutical composition according to claim 11 to a subject suffering from said disease or condition.

13. The method of claim 12, wherein said disease or condition is selected from depression, anxiety, schizophrenia, cognitive disorders, psychoses, obesity, inflammatory diseases, irritable bowel syndrome, inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, and testicular cancer.

14. The use of a compound in accord with Formula I: 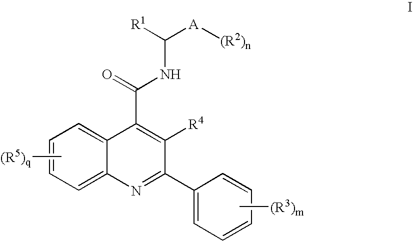
wherein:
R¹ is selected from H, C₁₋₄alkyl-, C₃₋₆cycloalkyl- and C₁₋₄alkylOC(O)-;
A is pyridyl;
R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;
n is 1, 2 or 3;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1, 2 or 3;
R⁴ is selected from H, —OH, —NH₂, —OSO₂R⁶, C₁₋₄alkyl-, C₁₋₄alkoxy-, C₁₋₆alkoxyC₁₋₆alkyl-, and E-(CH₂)ₚ-, where E is selected from —NR⁶R⁷, —SOC₁₋₆alkyl, —SO₂C₁₋₆alkyl, —NR⁶SO₂R⁷, —SR⁶, N⁺(O⁻)R⁶R⁷, aryl and an N- or C-linked 5- or 6-membered aromatic or non-aromatic heterocyclic ring having 1, 2, 3 or 4 nitrogen atoms or an N-oxide thereof, and p is 0, 1, 2, 3, 4 or 5;
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
and,
when R⁴ is E-(CH₂)ₚ- and said E thereof is an N or C linked 5- or 6-membered aromatic or non-aromatic heterocyclic ring or an N-oxide thereof, said E is unsubstituted or has 1, 2 or 3 substituents independently selected from —OH, ═O, —NH₂, —CN, halogen, C₁₋₄alkyl-, C₁₋₄alkoxy-, C₁₋₄alkyl-CO—, —NR⁶R⁷, aryl and a 5- or 6-membered aromatic or non-aromatic heterocyclic ring having 1, 2, 3 or 4 nitrogen atoms;
and,
when R¹, R², R³ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen;
or a stereoisomer, enantiomer, in vivo-hydrolysable precursor or pharmaceutically-acceptable salt thereof,
for the treatment or prophylaxis of a disease or condition in which modulation of the NK3 receptor is beneficial.

15. The use according to claim 14, wherein said disease or condition is selected from depression, anxiety, schizophrenia, cognitive disorders, psychoses, obesity, inflammatory diseases, irritable bowel syndrome, inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, and testicular cancer.

16. The use in the manufacture of a medicament for the treatment or prophylaxis of a disease or condition in which modulation of the NK3 receptor is beneficial of a compound in accord with Formula I: 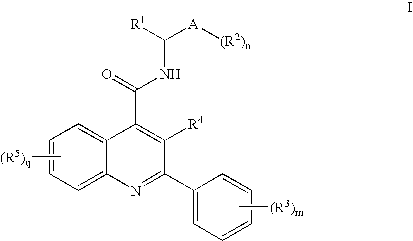
wherein:
R¹ is selected from H, C₁₋₄alkyl-, C₃₋₆cycloalkyl- and CI₄alkylOC(O)-;
A is pyridyl;
R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;
n is 1, 2 or 3;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1, 2 or 3;
R⁴ is selected from H, —OH, —NH₂, —OSO₂R⁶, C₁₋₄alkyl-, C₁₋₄alkoxy-, C₁₋₆alkoxyC₁₋₆alkyl-, and E-(CH₂)ₚ-, where E is selected from —NR⁶R⁷, —SOC₁₋₆alkyl, —SO₂C₁₋₆alkyl, —NR⁶SO₂R⁷, —SR⁶, N⁺(O⁻)R⁶R⁷, aryl and an N- or C-linked 5- or 6-membered aromatic or non-aromatic heterocyclic ring having 1, 2, 3 or 4 nitrogen atoms or an N-oxide thereof, and p is 0, 1, 2, 3, 4 or 5;
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
and,
when R⁴ is E-(CH₂)ₚ- and said E thereof is an N or C linked 5- or 6-membered aromatic or non-aromatic heterocyclic ring or an N-oxide thereof, said E is unsubstituted or has 1, 2 or 3 substituents independently selected from —OH, ═O, —NH₂, —CN, halogen, C₁₋₄alkyl-, C₁₋₄alkoxy-, C₁₋₄alkyl-CO—, —NR⁶R⁷, aryl and a 5- or 6-membered aromatic or non-aromatic heterocyclic ring having 1, 2, 3 or 4 nitrogen atoms;
and,
when R¹, R², R³ or R⁴ is an alkyl, cycloalkyl, alkoxy or alkoxyalkyl-moiety, said moieties are unsubstituted or have 1, 2, 3, 4 or 5 substituents independently selected at each occurrence from —OH, —NH₂, —CN, phenyl and halogen;
or a stereoisomer, enantiomer, in vivo-hydrolysable precursor or pharmaceutically-acceptable salt thereof.

17. The use according to claim 16, wherein said disease or condition is selected from depression, anxiety, schizophrenia, cognitive disorders, psychoses, obesity, inflammatory diseases, irritable bowel syndrome, inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, and testicular cancer.
